(19) United States
(12) Patent Application Publication
(10) Pub. No.: US 2006/0100689 A1
(43) Pub. Date: May 11, 2006

(54) Stent crimper with slit sheath

(75) Inventor: Jack Pryor, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/985,560

(22) Filed: Nov. 10, 2004

(51) Int. Cl. A61F 2/06

(52) U.S. Cl. 623/1.12

(57) ABSTRACT

The invention provides a system and method for crimping and sheathing a stent. The system comprises a radial compression device including a plurality of crimping members. Some or all of the crimping members include a contoured depression that receives and holds an inner sheath segment while the device moves between open and closed positions. In the method, a stent is positioned within the open compression device. The device is closed to crimp the stent and at least partially enclose the stent in the inner sheath segments. The crimped stent and inner sheath segments are slid into an outer sheath. The invention additionally provides a system for treating a vascular condition comprising a crimped stent at least partially enclosed within a segmented sheath member. The stent is simultaneously crimped and sheathed by segments of the sheath member held within contoured depressions formed in crimping members of a radial compression device.

### TECHNICAL FIELD

This invention relates generally to biomedical systems for treating vascular conditions and to systems and methods for manufacturing such biomedical systems. More specifically, the invention relates to a method and system for crimping a self-expandable stent while at the same time partially or wholly enclosing the stent in a slit sheath, and to a system for treating a vascular condition produced using the crimping and sheathing system and method.

### BACKGROUND OF THE INVENTION

Stents are cylindrical-shaped devices that are radially expandable to hold open a segment of a vessel or other anatomical lumen after implantation into the lumen. Various types of stents are in use, including expandable and self-expanding stents. Expandable stents generally are conveyed to the area to be treated on balloon catheters or other expandable devices. For a self-expanding stent, commonly a sheath is retracted, allowing expansion of the stent.

Stent insertion may cause undesirable reactions such as inflammation, infection, thrombosis, and proliferation of cell growth that occludes the passageway. Stents have been used with coatings to deliver drugs or other therapeutic agents at the site of the stent that may assist in preventing these conditions. The coatings must be bioengineered to control the release of highly potent and potentially toxic drugs.

Often a cap coating is deposited over one or more drug coatings to provide a carefully timed release of the drug(s). Protecting the cap coating from abrasion or other damage is important to ensure the desired delivery of the drug(s). Retracting a sheath from a self-expanding stent may scratch a coating deposited on the stent. Therefore, it would be desirable to have an improved system for treating a vascular condition and a manufacturing system and method for producing such a treatment system that overcomes the aforementioned and other disadvantages.

### SUMMARY OF THE INVENTION

One aspect of the present invention is a system for crimping and sheathing a stent. The system comprises a radial compression device including a plurality of crimping members. At least a plurality of the crimping members include a contoured depression. An inner sheath segment is received in each contoured depression. The depression holds the sheath segment while the compression device moves between an open and a closed position.

Another aspect of the present invention is a system for treating a vascular condition. The system comprises a crimped stent at least partially enclosed within a segmented sheath member. The stent was simultaneously crimped and sheathed by segments of the sheath member held within contoured depressions formed in crimping members of a radial compression device.

Yet another aspect of the present invention is a method of crimping and sheathing a stent. A plurality of inner sheath segments are positioned in contoured depressions formed in crimping members of a radial compression device. A stent is positioned within the radial compression device while the device is in an open position. The radial compression device is closed to crimp the stent and to at least partially enclose the stent in the inner sheath segments. The crimped stent and inner sheath segments are slid into an outer sheath.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIGS. 1A and 1B are illustrations of one embodiment of a system for crimping and sheathing a stent, in accordance with the present invention;

FIGS. 2A and 2B are isometric views of the system of FIGS. 1A and 1B;

FIGS. 3A and 3B are illustrations of another embodiment of a system for crimping and sheathing a stent, in accordance with the present invention, the system being capable of crimping a stent from a large expanded diameter to a small crimped diameter;

FIGS. 4A and 4B are illustrations of another embodiment of a system for crimping and sheathing a stent, in accordance with the present invention, each sheath segment being held in a shape lock depression within a crimping member;

FIGS. 5A and 5B are illustrations of another embodiment of a system for crimping and sheathing a stent, in accordance with the present invention, each sheath segment being held by an alternative shape lock depression within a crimping member;

FIGS. 6A and 6B are illustrations of another embodiment of a system for crimping and sheathing a stent, in accordance with the present invention, the crimping members shown as substantially triangular in cross section;

FIGS. 7A and 7B are illustrations of another embodiment of a system for crimping and sheathing a stent, in accordance with the present invention, each sheath segment having a spline to aid in holding the segment in a complementary contoured depression within a crimping member;

FIGS. 8A and 8B are illustrations of an embodiment of a system for treating a vascular condition, in accordance with the present invention; and

FIG. 9 is a flow diagram of one embodiment of a method of crimping and sheathing a stent, in accordance with the present invention.

### DETAILED DESCRIPTION OF THE PRESENTLY PREFERRED EMBODIMENTS

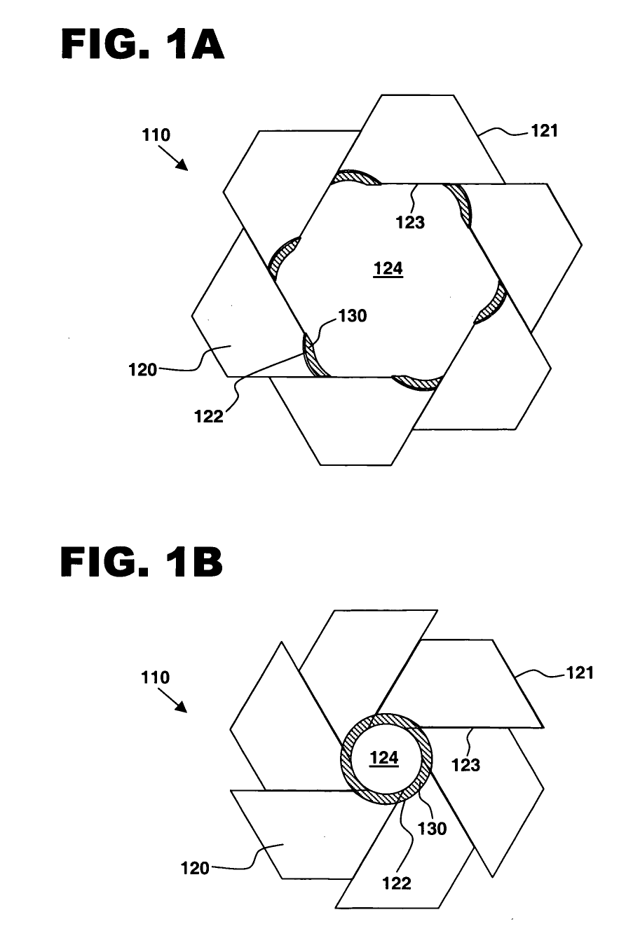

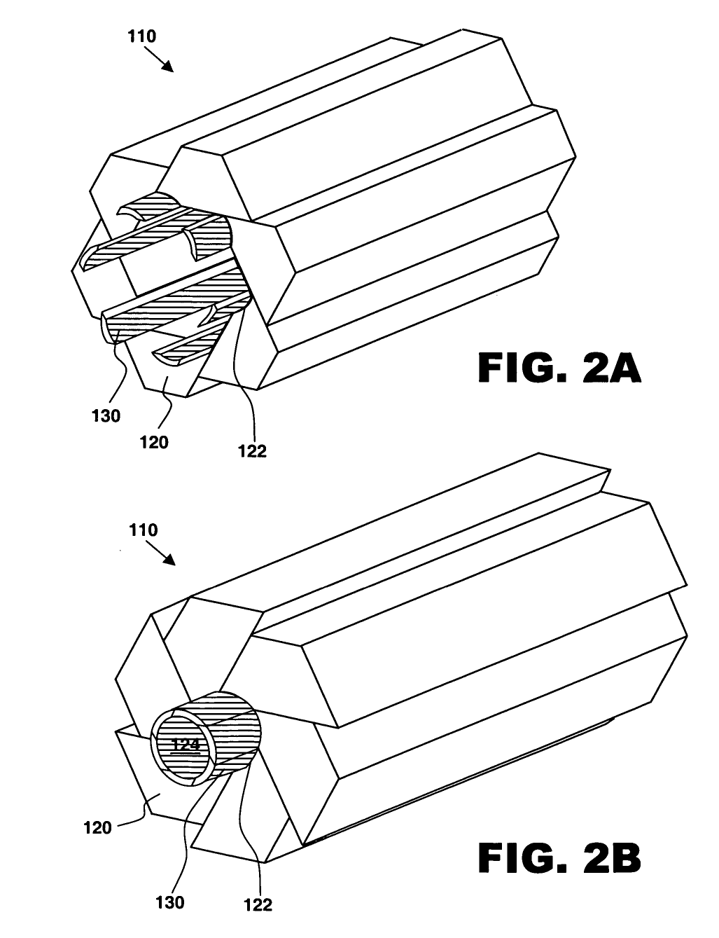

One aspect of the present invention is a system for crimping and sheathing a stent. One embodiment of the system, in accordance with the present invention, is illustrated in FIGS. 1A-2B. The system comprises a radial compression device 110 that includes multiple crimping members 120. Each crimping member includes a contoured depression 122. Inner sheath segments 130 are received in the contoured depressions.

As illustrated, radial compression device 110 includes six crimping members 120; however, the number of crimping members is variable. As is best seen in FIGS. 2A and 2B, each crimping member 120 is an elongated structure, with the length of the crimping member selected such that the member extends over the entire length of the stent to be crimped. In cross section, or as seen end-on as in FIGS. 1A and 1B, the crimping members are roughly trapezoidal, with a contoured depression 122 formed into each crimping member to hold an inner sheath segment 130. In another embodiment, not all of the crimping members may hold an inner sheath segment, with the remaining crimping members being designed to duplicate the shape formed by a crimping member plus a sheath segment.

Crimping members 120 may be formed from any appropriate material known in the art that is hard enough to crimp a stent and also formable to include a contoured depression. Such materials include, but are not limited to, stainless steel and a hard polymer. The material should be capable of being smoothed or polished to provide surfaces within contoured depressions 122 that allow inner sheath segments 130 to be easily removed from radial compression device 110 by sliding the segments out of the contoured depressions when the device is in a closed position.

Radial compression device 110 functions like a conventional variable diameter diaphragm, also called an iris diaphragm. In the present embodiment, crimping members 120 are arranged about the circumference of a circle with a side surface 121 of each crimping member in contact with a bottom surface 123 of an adjoining crimping member. The crimping members form a central aperture 124 having a variable diameter. Contoured depressions 122 of crimping members 120 face into this central aperture.

Each crimping member 120 rotates about a different longitudinal axis as compression device 110 moves between an open and a closed position. Rotation of the crimping members causes contoured depressions 122 to extend into central aperture 124 when the device moves from an open position to a closed position, thereby reducing the diameter of aperture 124 as the device closes.

Crimping members 120 form a central aperture sized to receive an expanded stent when compression device 110 is in an open position as seen in FIGS. 1A and 2A. The crimping members form a substantially cylindrical central aperture sized to crimp the stent when the compression device is in a closed position as seen in FIGS. 1B and 2B. As illustrated in FIG. 2B, sheath segments 130 held within contoured depressions 122 are positioned to fully enclose a crimped stent when compression device 110 is in a closed position. Sheath segments 130 are shown in FIGS. 2A and 2B as extending beyond crimping members 120 but may instead be fully contained within the compression device during operation of the device.

Sheath segments 130 may be manufactured from an appropriate biocompatible material, including, but not limited to, stainless steel, nitinol, polypropylene, polyethylene, a nylon/polyethylene blend, polytetrafluoroethylene (PTFE), a suitable polycarbonate, and combinations of the above. The segments may be formed by cutting evenly spaced longitudinal slits into a section of tubing, or each segment may be formed individually by, for example, extruding, molding, cutting, or stamping the segments. When cut from a section of tubing, the segments will be arc shaped in cross section. When formed by a method such as extruding or cutting, the segments may assume a variety of shapes in cross section, and may even be flat strips that when assembled form a substantially cylindrical structure.

Sheath segments 130 may be held within contoured depressions 122 by a variety of methods. For example, the sheath segments may be held by suction that is discontinued once compression device 110 has completed crimping and sheathing a stent, allowing the sheath segments and crimped stent to be easily removed from the compression device by sliding the sheathed stent out of the closed device. Alternatively, the sheath segment surface in contact with a contoured depression may be coated with a biocompatible material that lightly adheres to a crimping member while the compression device moves between an open position and a closed position and also serves as a lubricant to aid in sliding the sheath segment into and out of the crimping member. As another example, the sheath segments may be held by~ weak electrostatic attraction between the sheath segments and the crimping members that is produced by methods known in the art.

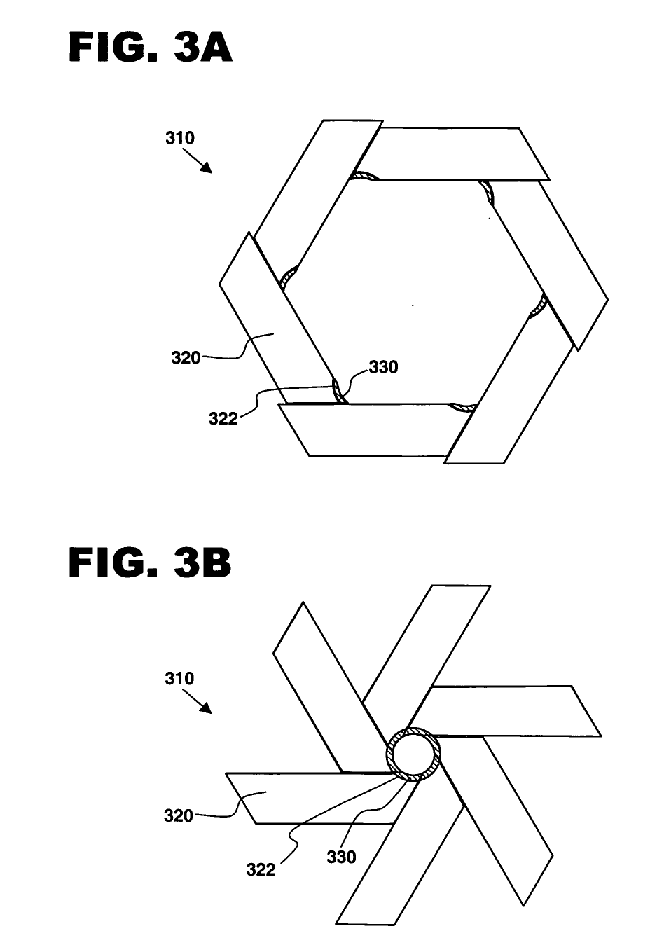

FIGS. 3A-7B show alternative embodiments in accordance with the present invention. The system shown open in FIG. 3A and closed in FIG. 3B is similar to that shown in FIGS. 1A-2B, having similarly shaped contoured depressions 322 and sheath segments 330, but with the dimensions of the depressions, segments, and crimping members 320 sized to permit a stent to be crimped from a larger expanded diameter to a smaller crimped diameter using compression device 310 than would be possible with compression device 110.

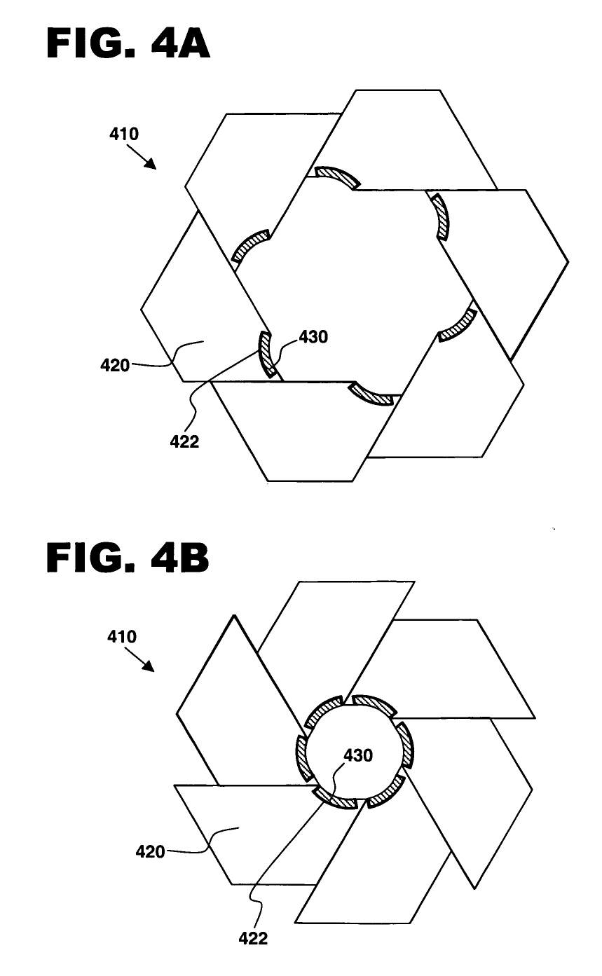

FIGS. 4A and 4B show a system in which contoured depressions 422 of crimping members 420 serve as shape locks for sheath segments 430. The sheath segments are held by the shape locks while compression device 410 moves between an open and a closed position but may be slidably removed from the device when the device is closed. In this example, sheath segments 430 are positioned to partially enclose a crimped stent when compression device 410 is in a closed position.

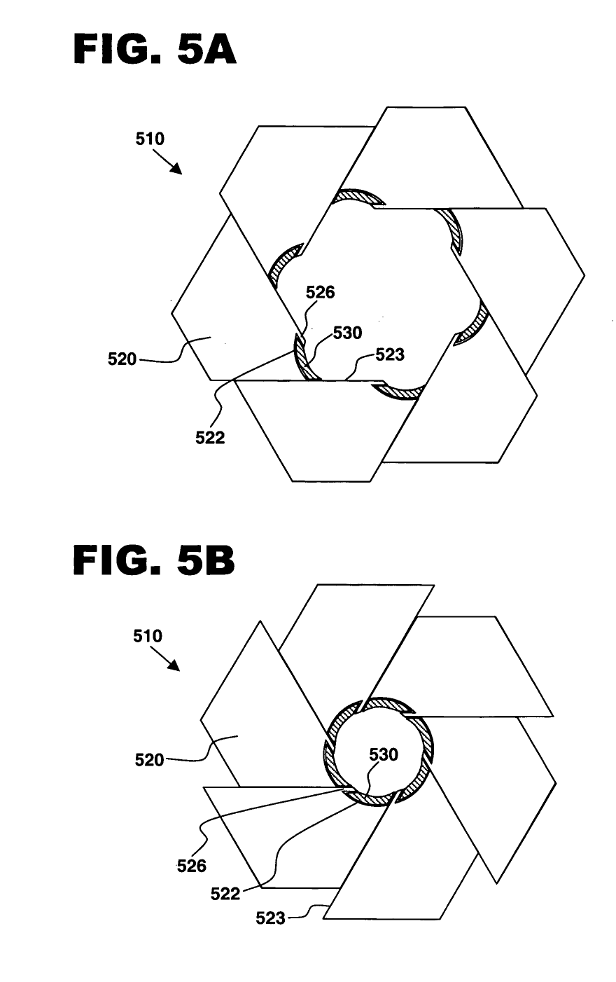

FIGS. 5A and 5B show another version of a shape lock. In this example, each sheath segment 530 is held within a contoured depression 522 by a combination of a tab 526 holding one side of the segment and the bottom surface 523 of the adjoining crimping member 520 abutting the opposite side of the segment, thereby holding the segments while compression device 510 moves from an open to a closed position. As in the system described above and illustrated in FIGS. 4A and 4B, sheath segments 530 are positioned to partially enclose a crimped stent when compression device 510 is in a closed position.

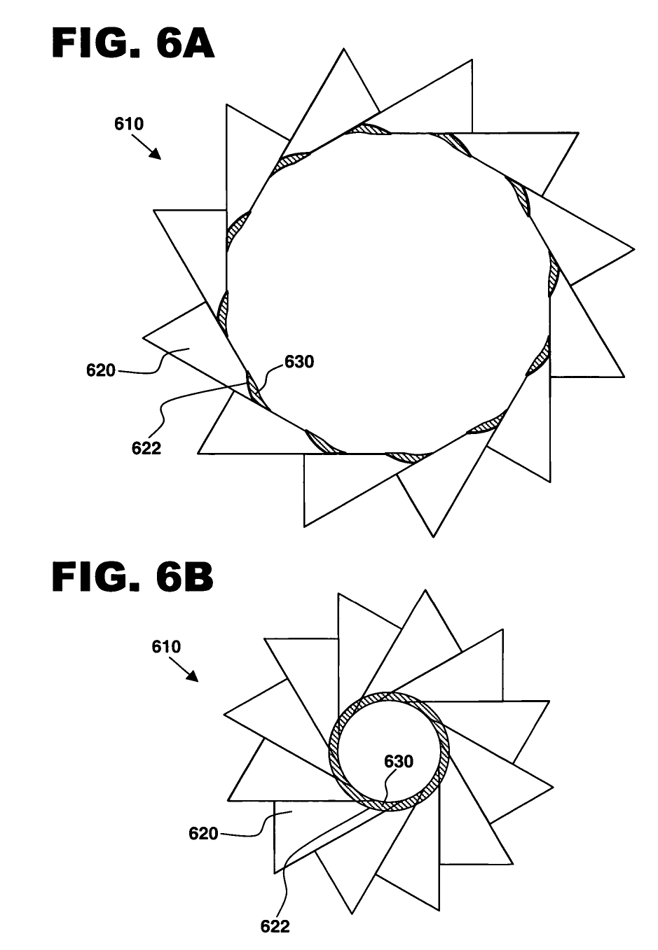

FIGS. 6A and 6B show a system with a compression device 610 having twelve crimping members 620 that are substantially triangular in cross section. As is illustrated by these figures, a system in accordance with the present invention may have various numbers of crimping members, and the crimping members need not be trapezoidal but may be any shape that permits sheath segments held within contoured depressions in the crimping members to be rotated into a central aperture formed by the crimping members, thereby reducing the radius of the aperture and simultaneously crimping and sheathing a stent. As illustrated in FIG. 6B, sheath segments 630 held within contoured depressions 622 are positioned to fully enclose a crimped stent when compression device 610 is in a closed position.

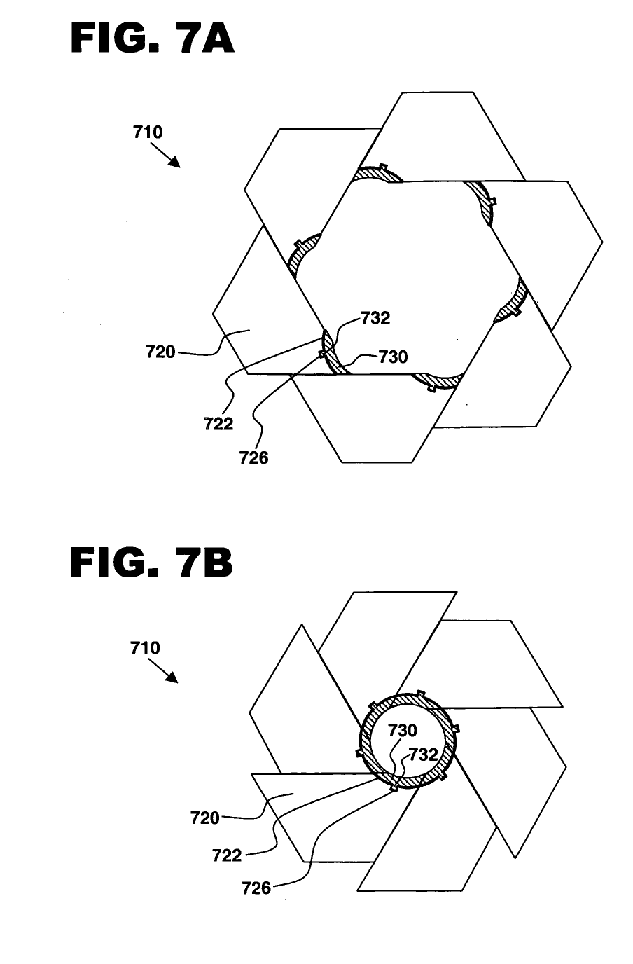

FIG. 7 shows a system having sheath segments 730 that include splines 732. The splines fit into complementary recesses 726 formed in contoured depressions 722. The splines aid in holding the segments as crimping members 720 rotate to transition compression device 710 between an open and a closed position.

In practice, the above-described systems are capable of crimping a stent in a substantially uniform manner along the full length of the stent. The invention allows stents of any diameter and length to be simultaneously crimped and at least partially enclosed within a segmented sheath. The sheath segments protect the crimped stent from abrasion as the stent is slid out of the compression device and offer the added advantage of allowing a crimped stent to be removed from the compression device by pulling on a proximal end of the segmented sheath rather than by pushing the sheathed stent out of the device. For example, a stent that does not have sufficient column strength or rigidity to be pushed out of the compression device may instead be pulled from the device, eliminating the risk of longitudinal compression or buckling of the stent. A combination of pushing on the stent and pulling on the segmented sheath may be employed if desired.

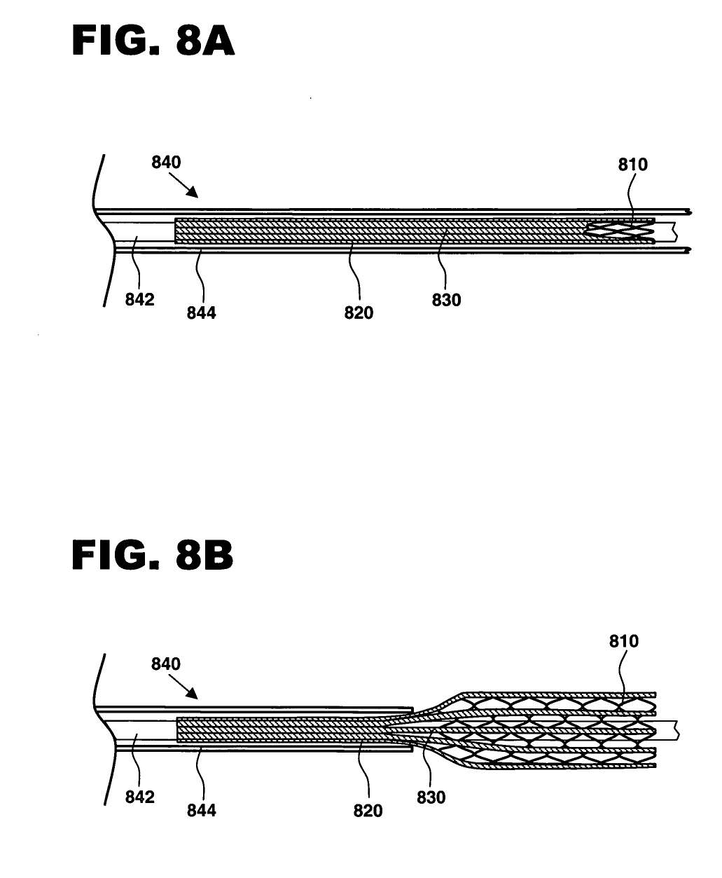

Another aspect of the present invention is a system for treating a vascular condition. One embodiment of the system, in accordance with the present invention, is illustrated in FIGS. 8A and 8B. The system comprises a crimped stent 810, a segmented sheath member 820 comprising multiple sheath segments 830, and a catheter 840 having an inner member 842 and an outer member 844.

In the present embodiment, crimped stent 810 is a coated, self-expanding, nitinol stent approximately 10 millimeters in expanded diameter and approximately 80 millimeters in length. However, the stent may be any size or design of self-expanding stent known in the art, manufactured from, for example, another nickel-titanium alloy, a nickel-cobalt alloy, another cobalt alloy, a thermoset plastic, stainless steel, a stainless steel alloy, or another biocompatible material.

Crimped stent 810 is shown in FIG. 8A fully enclosed within segmented sheath member 820. The stent is simultaneously crimped and sheathed by segments 830 of sheath member 820 held within contoured depressions formed in crimping members of a radial compression device. Such a device is described above and illustrated in FIGS. 1A-7B. Full enclosure of the crimped stent may be accomplished using a crimping and sheathing system such as is seen in FIGS. 1A-2B, 3A and 3B, 6A and 6B, and 7A and 7B. In another embodiment, the sheath segments may partially enclose the crimped stent as would result when using a system such as that seen in FIGS. 4A and 4B, and 5A and 5B.

As illustrated in FIGS. 8A and 8B, sheath member 820 comprises six sheath segments 830; however, the number of sheath segments is variable. For the present embodiment, sheath member 820 is preferably formed from one or more materials nonreactive with a coated stent, for example polypropylene, polyethylene, a nylon/polyethylene blend, PTFE (polytetrafluoroethylene), or a suitable polycarbonate. In another embodiment, the sheath member may comprise another appropriate biocompatible material, including, but not limited to, stainless steel and nitinol. Sheath segments 830 may be formed by cutting evenly spaced longitudinal slits into a section of tubing, or each segment may be formed individually by, for example, extruding, molding, cutting, or stamping the segments.

In the present embodiment, stent 810 is delivered to a treatment site using catheter 840, which includes inner member 842 and outer member 844. Stent 810 is crimped about inner member 842. Outer member 844 is drawn over both stent 810 and sheath member 820 during manufacture-of the system and serves as an outer sheath that holds stent 810 in a compressed configuration and maintains sheath segments 830 in place about the crimped stent.

As shown in FIGS. 8A and 8B, sheath member 820 is longer than the stent it partially encloses but substantially shorter than inner member 842. In the present embodiment, a proximal portion of sheath member 820 is attached to a distal portion of inner member 842 using, for example, an adhesive or heat bonding. Where sheath segments 830 are cut from a section of tubing, an uncut proximal portion of the tubing may be attached to the inner member. Where the sheath segments are formed individually, they may be individually attached to the inner member. In another embodiment, sheath member 820 may be approximately the same length as inner member 842, with sheath segments 830 formed into the distal end of the sheath member. In this embodiment, the sheath member is not attached to the inner member and is held in position relative to the inner member by a handle attached to a proximal end of the system.

FIG. 8A shows the system ready for delivery into the vascular system of a patient. Outer member 844 is shown in cross section. Inner member 842 and sheath member 820 are shown in full except for a portion of sheath member 820 that is cut away to reveal crimped stent 810. Sheath segments 830 protect crimped stent 810 from abrasion as outer member 844 is withdrawn to deliver stent 810 at the treatment site. The sheath segments also hold stent 810 in position about inner member 842 as outer member 844 is withdrawn, protecting the stent from being longitudinally compressed or otherwise distorted as the outer member passes over the stent. One skilled in the art will appreciate that stent 810 may be deployed by extending inner member 842 or by pushing on a proximal end of stent 810 in combination with or instead of withdrawing outer member 844. A stent experiences less force during deployment using the present invention than using a conventional delivery system because the segmented sheath effectively reinforces the stent.

FIG. 8B shows the system partially deployed, with outer member 844 partially retracted. Once outer member 844 has been retracted, stent 810 expands, thereby expanding the portion of sheath member 820 enclosing the stent. As sheath member 820 expands, sheath segments 830 separate and are pressed against the wall of the target vessel by the expanded stent. Sheath member 820 is preferably segmented such that the sheath segments extend proximal to the proximal edge of crimped stent 810. For example, where the stent is approximately 80 millimeters in length, the sheath member may be segmented to a point approximately 1 centimeter proximal to the proximal edge of the stent. This allows sheath segments 830 to fully separate along the entire length of the stent, thereby allowing crimped stent 810 to expand along the entire length of the stent once outer member 844 has been fully retracted.

Once stent 810 has fully expanded, catheter 840 may be removed from the patient, simultaneously withdrawing inner member 842 from within stent 810 and sheath member 820 from the outside of stent 810, leaving the stent in place within the vessel. Because the separated sheath segments cover only a fraction of the outer surface of the expanded stent and exert only as much pressure on the stent as is produced by the expansive force of the stent against the inner wall of the vessel, little or no damage is caused to the stent or stent coating as the sheath segments are withdrawn over the outer surface of the expanded stent.

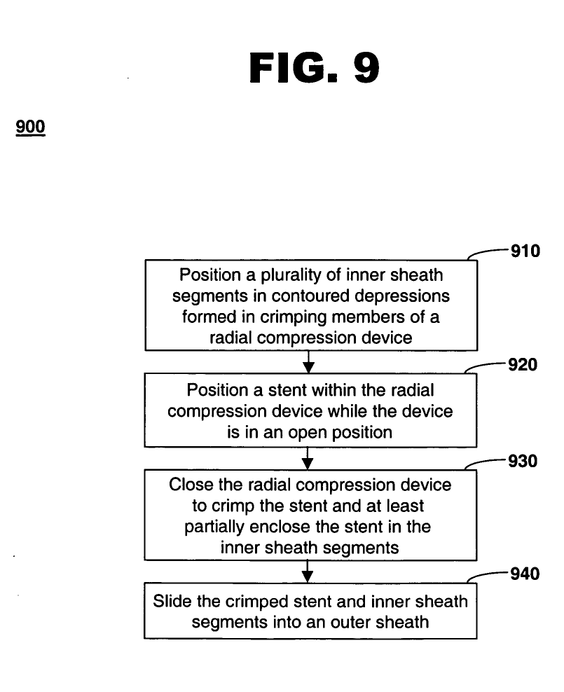

Yet another aspect of the present invention is a method of crimping and sheathing a stent. FIG. 9 shows a flow diagram of one embodiment of the method in accordance with the present invention.

A plurality of inner sheath segments are positioned in contoured depressions formed in crimping members of a radial compression device (Block 910). The radial compression device may be one such as has been described above and illustrated in FIGS. 1A-7B, with the crimping members forming a central aperture into which an expanded stent is inserted for crimping.

The sheath segments may be positioned in the contoured depressions either simultaneously or individually. In the present embodiment, prior to inserting the segments into the device, the segments are attached adjacent to a distal end of a catheter inner member. In this embodiment, the segments are slid into the contoured depressions as a group while the compression device is either open or closed, at the same time positioning a distal portion of the inner member within the central aperture formed by the crimping members. Where the device is closed during positioning of the segments, it must be opened prior to positioning a stent within the device.

Alternatively, the sheath segments may be positioned within the contoured depressions, either simultaneously or individually, while the device is open, and a catheter inner member may be separately positioned within the central aperture of the compression device. The segments may be attached to the inner member when the compression device is closed to crimp a stent. In still another alternative, the inner sheath segments may be formed into a distal portion of an elongated section of tubing, and an inner member may be threaded into the tubing either before or after the sheath segments are positioned in the contoured depressions of the crimping members.

A stent is positioned within the radial compression device while the device is in an open position (Block 920). In the present embodiment, an expanded stent is inserted into the device over the distal portion of the inner member that has already been positioned within the central aperture of the compression device. The stent is thereby positioned within the sheath segments, which when held by the crimping members form a roughly cylindrical structure. Thus, the sheath segments, the stent, and the inner member are all positioned coaxial to one another, with the sheath segments forming the outer layer, the catheter inner member forming the inner layer, and the stent sandwiched between. One skilled in the art will appreciate that the order of placing the sheath segments, the inner member, and the stent into the compression device may be varied; however, the order described above is believed to be the most efficient.

The radial compression device is closed to crimp the stent and to at least partially enclose the stent in the inner sheath segments (Block 930). As previously described, the contoured depressions and the sheath segments they hold are rotated into the central aperture of the compression device to reduce the diameter of the aperture, thereby crimping the stent. At the same time, the sheath segments are positioned around the crimped stent, thereby at least partially enclosing the stent. The degree of enclosure will depend upon the design of the individual crimping members and the percentage of crimping members in the compression device that hold sheath segments.

The crimped stent and inner sheath segments are slid into an outer sheath (Block 940). In the present embodiment, the sheath segments, crimped stent, and catheter inner member are simultaneously withdrawn from the closed compression device into a proximal portion of the catheter outer member. The outer member serves as an outer sheath to hold the stent in a compressed configuration and the sheath segments in place about the crimped stent.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for crimping and sheathing a stent, comprising:
a radial compression device including a plurality of crimping members, at least a plurality of the crimping members each including at least one contoured depression; and
a plurality of inner sheath segments received in the contoured depressions;
wherein the contoured depressions hold the sheath segments while the compression device moves between an open position and a closed position.

2. The system of claim 1 wherein the crimping members are disposed about the circumference of a circle.

3. The system of claim 1 wherein each crimping member rotates about a different longitudinal axis as the radial compression device moves between an open and a closed position.

4. The system of claim 1 wherein as the radial compression device moves from an open position to a closed position, the contoured depression of each crimping member extends into a central aperture formed by the crimping members.

5. The system of claim 1 wherein the crimping members form a central aperture sized to receive an expanded stent when the radial compression device is in an open position, and wherein the crimping members form a substantially cylindrical central aperture sized to crimp the stent when the radial compression device is in a closed position.

6. The system of claim 1 wherein each contoured depression serves as a shape lock to hold a sheath segment.

7. The system of claim 1 wherein the sheath segments are held within the contoured depressions using a method selected from a group consisting of suction, adhesion, and electrostatic attraction.

8. The system of claim 1 wherein the sheath segments are positioned to at least partially enclose a crimped stent when the radial compression device is in a closed position.

9. The system of claim 1 wherein the contoured depressions allow the sheath segments to be slidably removed from the radial compression device when the device is in a closed position.

10. The system of claim 1 wherein each crimping member is substantially trapezoidal in cross section.

11. The system of claim 1 wherein each crimping member is substantially triangular in cross section.

12. A system for treating a vascular condition, comprising:
a crimped stent at least partially enclosed within a segmented sheath member, wherein the stent was simultaneously crimped and sheathed by segments of the sheath member held within contoured depressions formed in crimping members of a radial compression device.

13. The system of claim 12, further comprising:
a catheter having an inner member and an outer member.

14. The system of claim 13 wherein the stent is crimped about the inner member.

15. The system of claim 13 wherein the sheath member is attached to the inner member.

16. The system of claim 13 wherein the outer member serves as an outer sheath.

17. A method of crimping and sheathing a stent, the method comprising:
positioning a plurality of inner sheath segments in contoured depressions formed in crimping members of a radial compression device;
positioning a stent within the radial compression device while the device is in an open position;
closing the radial compression device to crimp the stent and to at least partially enclose the stent in the inner sheath segments; and
sliding the crimped stent and inner sheath segments into an outer sheath.

18. The method of claim 17, the method further comprising:
closing the radial compression device prior to positioning the inner sheath segments in the contoured depressions formed in the crimping members; and
opening the radial compression device prior to positioning the stent within the radial compression device.

19. The method of claim 17 wherein the plurality of inner sheath segments are attached adjacent to a distal end of an inner member, and wherein positioning a plurality of inner sheath segments in contoured depressions formed in crimping members of a radial compression device further comprises positioning a distal portion of the inner member within a central aperture of the radial compression device.

20. The method of claim 19 wherein positioning a stent within the open radial compression device further comprises positioning the stent over the distal portion of the inner member.
